(19) United States
(12) Patent Application Publication
(10) Pub. No.: US 2025/0143737 A1
(43) Pub. Date: May 8, 2025

(54) SURGICAL INSTRUMENT

(71) Applicant: LIVSMED INC., Seongnam-si (KR)

(72) Inventors: Dong Hoon KANG, Seoul (KR); Tae Jin PARK, Seongnam-si (KR); Hyun JOO, Seoul (KR); Ki Woong KIM, Suwon-si (KR); Mi Ryoung CHOI, Seongnam-si (KR); Jae Yeong LEE, Seongnam-si (KR)

(21) Appl. No.: 18/934,829

(22) Filed: Nov. 1, 2024

(30) Foreign Application Priority Data
Nov. 3, 2023 (KR) 10-2023-0151160

(51) Int. Cl. A61B 17/29; A61B 17/00

(52) U.S. Cl. CPC A61B 17/2909; A61B 2017/00477; A61B 2017/00862; A61B 2017/2912; A61B 2017/2925; A61B 2017/2929; A61B 2017/2932

(57) ABSTRACT

A surgical instrument includes an end tool including a first jaw and a second jaw that are rotatable, a manipulation part including an actuation manipulation part that controls actuation motions of the first jaw and the second jaw, and a power transmission part including a first jaw wire and a second jaw wire connected to the manipulation part to transfer the rotation of the manipulation part to the first jaw and the second jaw, respectively, wherein the actuation manipulation part includes an actuation pulley configured to be rotatable around an actuation rotation shaft, and a handle member that is fixedly coupled to the actuation pulley and rotated along with the actuation pulley, and a tensioner that rotates along with the rotation of the actuation pulley, and the tensioner comes into contact with the first jaw wire or the second jaw wire according to the rotation of the actuation pulley, and applies additional tension to the first jaw wire or the second jaw wire.

### CROSS-REFERENCE TO RELATED APPLICATION

This application is based on and claims priority under 35 U.S.C. § 119 to Korean Patent Application No. 10-2023-0151160, filed on Nov. 3, 2023, in the Korean Intellectual Property Office, the disclosure of which is incorporated by reference herein in its entirety.

### BACKGROUND

### 1. Field

The present disclosure relates to a surgical instrument, and more specifically, to a surgical instrument that may be manually operated to be used in laparoscopic surgery or other various surgeries.

### 2. Description of the Related Art

Surgery denotes a process of curing illness by cutting, incising, or manipulating the skin, the mucosa layer, and other tissues by using a medical instrument. In particular, laparotomy that treats, shapes, or removes an organ by cutting and opening the skin of a surgical site may cause bleeding, side effects, pain of a patient, scar, etc. Therefore, surgery performed by inserting only a medical instrument, e.g., a laparoscope, a surgical instrument, a microscope for microsurgery, etc. after forming a predetermined hole in the skin, or surgery using a robot has been recently considered as an alternative.

A surgical instrument is an instrument for carrying out surgery on a surgical site by manipulating an end tool provided at an end of a shaft that passes through a hole in the skin, manually by a doctor with his/her own hands or by using a robot arm. The end tool provided at the surgical instrument performs a pivoting motion, a gripping motion, a cutting motion, etc. via a predetermined structure.

The above-mentioned background technology is technical information that the inventor possessed for deriving the present invention or acquired in the process of deriving the present invention, and cannot necessarily be said to be known art disclosed to the general public before filing the application for the present invention.

### SUMMARY

The present disclosure is directed to providing a surgical instrument which may be operatable manually to be used in laparoscopic surgery or other various surgeries and may have an improved usability by maintaining motion integrity between a manipulator and an end tool and amplifying a grip force during a maximum grip.

Additional aspects will be set forth in part in the description which follows and, in part, will be apparent from the description, or may be learned by practice of the presented embodiments of the disclosure.

According to an embodiment of the present disclosure, a surgical instrument includes an end tool including a first jaw and a second jaw that are configured to be rotatable, a manipulation part including an actuation manipulation part that controls actuation motions of the first jaw and the second jaw, and configured to control motions of the end tool, and a power transmission part including a first jaw wire connected to the manipulation part to transfer the rotation of the manipulation part to the first jaw, and a second jaw wire connected to the manipulation part to transfer the rotation of the manipulation part to the second jaw, wherein the actuation manipulation part includes an actuation pulley configured to be rotatable around an actuation rotation shaft, and a handle member that is fixedly coupled to the actuation pulley and rotated along with the actuation pulley, and a tensioner that rotates along with the rotation of the actuation pulley, and the tensioner comes into contact with the first jaw wire or the second jaw wire according to the rotation of the actuation pulley, and applies additional tension to the first jaw wire or the second jaw wire.

In the present embodiment, the tensioner may be fixedly coupled to the actuation pulley.

In the present embodiment, the tensioner may be formed integrally with the actuation pulley, and the actuation pulley may be a non-circular pulley.

In the present embodiment, the tensioner may be formed on the handle member.

In the present embodiment, the end tool may further include an end tool jaw pulley that is coupled to the first jaw or the second jaw and configured to be rotatable around a jaw rotation shaft, the end tool jaw pulley and the actuation pulley may be connected to each other via at least one jaw wire between the first jaw wire and the second jaw wire, the end tool jaw pulley may have a diameter that is less than a diameter of the actuation pulley, and when the at least one jaw wire moves, a rotation angle of the end tool jaw pulley may be greater than a rotation angle of the actuation pulley.

In the present embodiment, with respect to the rotation angle of the end tool jaw pulley according to the rotation of the actuation pulley, when the rotation angle of the end tool jaw pulley according to the rotation of the actuation pulley in a state in which the tensioner comes into contact with the first jaw wire or the second jaw wire is defined as a first rotation angle, and when the rotation angle of the end tool jaw pulley according to the rotation of the actuation pulley in a state in which the tensioner is spaced apart from the first jaw wire or the second jaw wire is defined as a second rotation angle, the first rotation angle may be greater than the second rotation angle.

In the present embodiment, the actuation manipulation part may include a first actuation manipulation part including a first actuation pulley configured to be rotatable around a first actuation rotation shaft, and a first handle member fixedly coupled to the first actuation pulley and rotated along with the first actuation pulley, and a second actuation manipulation part including a second actuation pulley configured to be rotatable around a second actuation rotation shaft, and a second handle member fixedly coupled to the second actuation pulley and rotated along with the second actuation pulley.

In the present embodiment, the end tool may have a closed state in which the first jaw and the second jaw are in contact with each other and an open state in which the first jaw and the second jaw are spaced apart from each other, the actuation manipulation part may have an input angle defined as an angle relationship between the first handle member and the second handle member, when the actuation manipulation part has an input angle greater than or equal to a first angle determined in advance, the end tool may have the open state, and when the actuation manipulation part has an input angle less than the first angle determined in advance, a pressure between the first jaw and the second jaw may be changed.

In the present embodiment, when the actuation manipulation part has an input angle greater than the first angle, the tensioner may not be in contact with the first jaw wire or the second jaw wire, and when the actuation manipulation part has an input angle less than the first angle, the tensioner may be in contact with the first jaw wire or the second jaw wire.

In the present embodiment, when the actuation manipulation part has an input angle that is greater than or equal to the first angle determined in advance, at least one of the first jaw wire and the second jaw wire may not be elastically deformed, and when the actuation manipulation part has an input angle that is less than the first angle determined in advance, at least one of the first jaw wire and the second jaw wire may be elastically deformed.

In the present embodiment, when the actuation manipulation part has an input angle less than the first angle determined in advance, the input angle may be greater than or equal to a second angle determined in advance so as to prevent plastic deformation of at least one of the first jaw wire and the second jaw wire.

In the present embodiment, the second angle may be formed in a state in which the first handle member and the second handle member are in contact with each other.

In the present embodiment, when the actuation manipulation part has an input angle less than the first angle determined in advance, a force applied to the first jaw wire and the second jaw wire may be greater than a force applied to the first jaw wire and the second jaw wire when the actuation manipulation part has an input angle greater than the first angle determined in advance.

In the present embodiment, the end tool may further include a grip state in which a first contact surface of the first jaw and a second contact surface of the second jaw are in contact with an external object, when the actuation manipulation part has an input angle that is greater than or equal to the second angle, the end tool may have an open state, and an angle between the first contact surface and the second contact surface may be changed due to a rotation of at least one of the first handle member and the second handle member, and when the actuation manipulation part has an input angle less than the second angle, the end tool may maintain the grip state, and a pressure between the first contact surface and the second contact surface and the external object may be changed due to a user input causing a rotation of at least one of the first handle member and the second handle member.

Other aspects, features and advantages other than those described above will become apparent from the following detailed description of the drawings, claims and disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating a surgical instrument according to a first embodiment of the present disclosure;

FIG. 2 is a perspective view illustrating an end tool of the surgical instrument of FIG. 1;

FIGS. 3 and 4 are perspective views illustrating a manipulation part of the surgical instrument of FIG. 1;

FIGS. 5A and 5B are diagrams showing a grip-press mechanism of the surgical instrument according to the first embodiment of the present disclosure in view of a relationship between a manipulation part and an end tool;

FIGS. 6A and 6B are diagrams showing an object press mechanism of the surgical instrument according to the first embodiment of the present disclosure in view of a relationship between a manipulation part and an end tool;

FIG. 7 is a diagram schematically showing a configuration in order to describe motions of the end tool and the manipulation part of the surgical instrument of FIG. 1;

FIG. 8 is a plan view showing a comparative technique for illustrating an actuation manipulation part of the surgical instrument according to the first embodiment of the present disclosure;

FIG. 9 is a plan view showing the actuation manipulation part of the surgical instrument according to the first embodiment of the present disclosure; and

FIG. 10 is a plan view showing an actuation manipulation part of a surgical instrument according to a modified example of the first embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The embodiments will be described below in more detail with reference to the accompanying drawings. Those components that are the same or are in correspondence are rendered the same reference numeral regardless of the figure number, and redundant explanations are omitted.

As the present disclosure allows for various changes and numerous embodiments, particular embodiments will be illustrated in the drawings and described in detail in the written description. The attached drawings for illustrating one or more embodiments are referred to in order to gain a sufficient understanding, the merits thereof, and the objectives accomplished by the implementation. However, the embodiments may have different forms and should not be construed as being limited to the descriptions set forth herein.

In the description, certain detailed explanations of the related art are omitted when it is deemed that they may unnecessarily obscure the essence of the present disclosure.

An expression used in the singular encompasses the expression of the plural, unless it has a clearly different meaning in the context. It will be understood that although the terms “first,” “second,” etc. may be used herein to describe various components, these components should not be limited by these terms. These components are only used to distinguish one component from another. These components are only used to distinguish one component from another.

In the present specification, it is to be understood that the terms such as “including,” “having,” and “comprising” are intended to indicate the existence of the features or components disclosed in the specification, and are not intended to preclude the possibility that one or more other features or components may exist or may be added.

It will be understood that when a unit, region, or component is referred to as being “formed on” another layer, region, or component, it can be directly or indirectly formed on the other layer, region, or component. That is, for example, intervening units, regions, or components may be present.

It will be understood that when an element is referred to as being “connected” or “coupled” to another element, it can be directly connected or coupled to the other element or intervening elements may be present.

Sizes of components in the drawings may be exaggerated for convenience of explanation. In other words, since sizes and thicknesses of components in the drawings are arbitrarily illustrated for convenience of explanation, the following embodiments are not limited thereto.

A surgical instrument according to the present disclosure is characterized in that, with respect to an actuation motion, when a manipulation part is rotated, an end tool is rotated in a direction that is intuitively the same as a manipulation direction of the manipulation part. The description of the present disclosure is provided based on the actuation motion of the surgical instrument, but the present disclosure is not limited thereto, and the surgical instrument may perform at least one of a pitch motion and a yaw motion. Also, the surgical instrument capable of performing such above composite motions according to the present disclosure is characterized in that, when a manipulation part is rotated in a direction with respect to at least one of the pitch motion, the yaw motion, and the actuation motion, the end tool is intuitively rotated in a direction that is the same as the manipulation direction of the manipulation part.

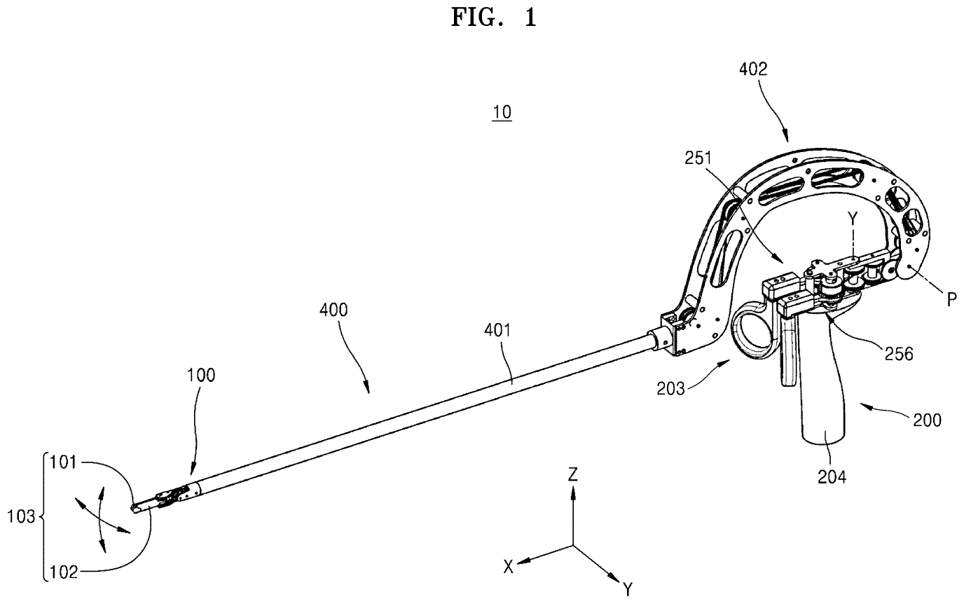

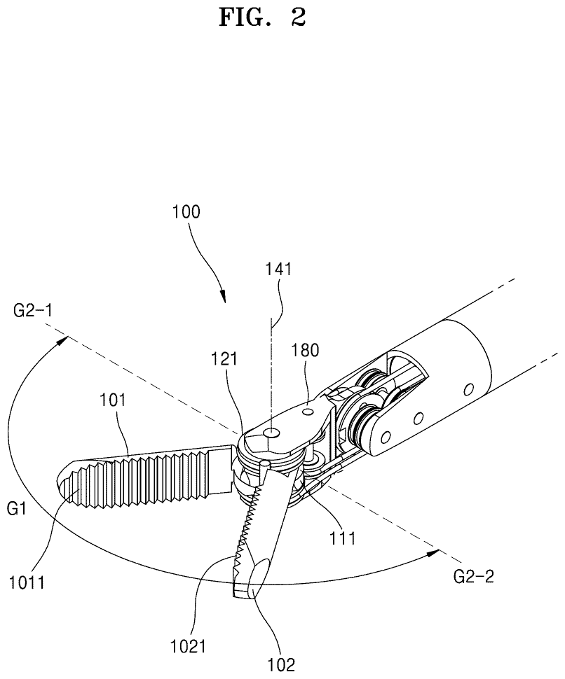

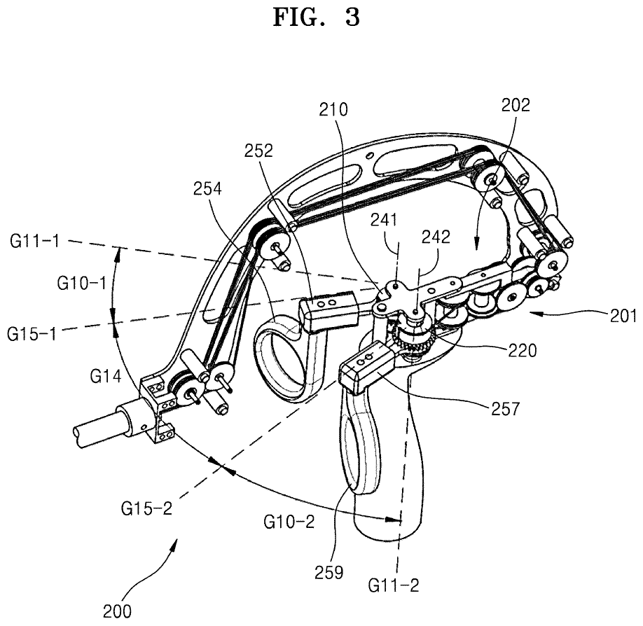

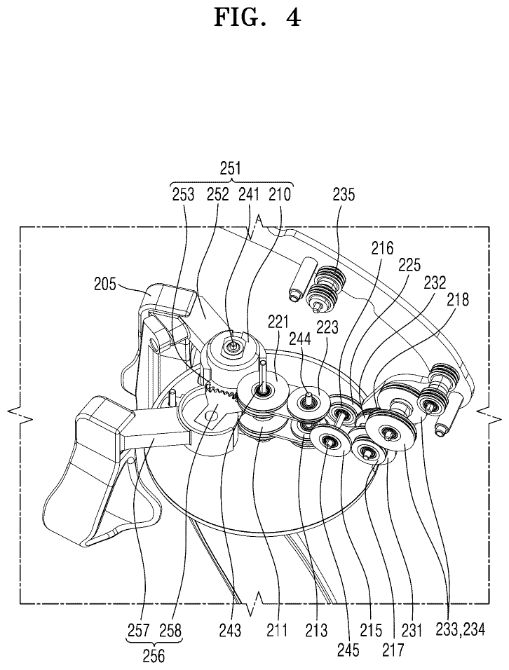

FIG. 1 is a perspective view showing a surgical instrument according to a first embodiment of the present disclosure, FIG. 2 is a perspective view showing an end tool of the surgical instrument of FIG. 1, and FIGS. 3 and 4 are perspective views showing a manipulation part of the surgical instrument of FIG. 1.

First, referring to FIG. 1, a surgical instrument 10 according to the first embodiment of the present disclosure includes an end tool 100, a manipulation part 200, a power transmission part (not shown), and a connection part 400.

Here, the connection part 400 is formed in the shape of a hollow shaft, and one or more wires or electric wires may be accommodated therein. The manipulation part 200 is coupled to one end portion of the connection part 400, the end tool 100 is coupled to the other end portion of the connection part 400, and thus, the connection part 400 may serve to connect the manipulation part 200 and the end tool 100.

Here, the connection part 400 of the surgical instrument 10 according to the first embodiment of the present disclosure includes a straight part 401 and a bent part 402, the straight part 401 is formed at a side of the connection part 400 coupled to the end tool 100, and the bent part 402 may be formed at a side of the connection part 400, to which the manipulation part 200 is coupled. As such, since the end portion of the connection part 400 at the side of the manipulation part 200 is formed to be bent, an actuation manipulation part 203 may be formed along an extension line of the end tool 100 or adjacent to the extension line. From another perspective, it may be said that the manipulation part 200 is at least partially accommodated in a concave portion formed by the bent part 402. Due to the above-described shape of the bent part 402, the shapes and motions of the manipulation part 200 and the end tool 100 may be further intuitively matched with each other.

The manipulation part 200 is formed at the one end portion of the connection part 400 and provided as an interface to be directly controlled by a medical doctor, for example, a tongs shape, a stick shape, a lever shape, or the like, and when the medical doctor controls the manipulation part 200, the end tool 100, which is connected to the interface and inserted into the body of a surgical patient, performs a certain motion, thereby performing surgery. Here, the manipulation part 200 is illustrated in FIG. 3 as being formed in a handle shape that is rotatable while the finger is inserted therein, but the concept of the present disclosure is not limited thereto, and various types of manipulation parts that may be connected to the end tool 100 and manipulate the end tool 100 may be possible.

The end tool 100 is formed on the other end portion of the connection part 400, and performs necessary motions for surgery by being inserted into a surgical site. As an example of the end tool 100 described above, a pair of jaws 103 for performing a grip motion may be used as illustrated in FIG. 2. However, the concept of the present disclosure is not limited thereto, and various devices for performing surgery may be used as the end tool 100. For example, a configuration of a cantilever cautery may also be used as the end tool. The end tool 100 is connected to the manipulation part 200 via a power transmission part and receives a driving force of the manipulation part 200 through the power transmission part, and thus performs a motion necessary for surgery such as gripping, cutting, suturing, or the like.

Here, the end tool 100 of the surgical instrument 10 according to the first embodiment of the present disclosure is formed to be rotatable in one or more directions, for example, the end tool 100 may be formed to perform a pitch motion around a Y-axis of FIG. 1 and simultaneously perform a yaw motion and an actuation motion around a Z-axis of FIG. 1.

Here, the pitch motion, the yaw motion, and the actuation motion used in the present disclosure are defined as follows.

First, the pitch motion denotes a motion of the end tool 100 rotating in a vertical direction with respect to an extension direction of the connection part 400 (an X-axis direction of FIG. 1), that is, a motion rotating around the Y-axis of FIG. 1. In other words, the pitch motion denotes a motion of the end tool 100, which is formed to extend from the connection part 400 in the extension direction of the connection part 400 (the X-axis direction of FIG. 1), rotating vertically around the Y-axis with respect to the connection part 400.

Next, the yaw motion denotes a motion of the end tool 100 rotating in left and right directions with respect to the extension direction of the connection part 400 (the X-axis direction of FIG. 1), that is, a motion rotating around the Z-axis of FIG. 1. In other words, the yaw motion denotes a motion of the end tool 100, which is formed to extend from the connection part 400 in the extension direction of the connection part 400 (the X-axis direction of FIG. 1), rotating left and right around the Z-axis with respect to the connection part 400. That is, the yaw motion denotes the rotation of two jaws 103 formed at the end tool 100 in the same direction around the Z-axis.

In addition, the actuation motion denotes a folding or unfolding motion of two jaws 103 rotating in opposite directions, while the end tool 100 rotates around the same rotation shaft as that of the yaw motion. That is, the actuation motion denotes the rotation of two jaws 103 formed at the end tool 100 in the opposite directions around the Z-axis.

That is, a first jaw and a second jaw may rotate around a rotation shaft 141 independently of each other while changing an angle therebetween. Here, the independent rotation of the first jaw and the second jaw while changing the angle therebetween may be referred to as a ‘grip motion’.

The power transmission part serves to connect the manipulation part 200 to the end tool 100 and transmit the driving force of the manipulation part 200 to the end tool 100, and may include a plurality of wires, pulleys, links, sections, gears, or the like.

Hereinafter, intuitive driving of the surgical instrument 10 of the present disclosure will be described.

First, a user may perform a pitch motion by rotating a first handle 204 around the Y-axis (that is, rotation shaft P) while holding the first handle 204 with the palm of the hand, and may perform a yaw motion by rotating the first handle 204 around the Z-axis (that is, rotation shaft Y). In addition, the user may perform an actuation motion by manipulating the actuation manipulation part 203 while inserting the thumb and the index finger into a first handle member 252 and/or a second handle member 257 in the form of a hand ring formed at one end portion of the actuation manipulation part 203.

Here, in the surgical instrument 10 according to the first embodiment of the present disclosure, when the manipulation part 200 is rotated in one direction with respect to the connection part 400, the end tool 100 may be rotated in a direction that is intuitively the same as a manipulation direction of the manipulation part 200. In other words, when the first handle 204 of the manipulation part 200 is rotated in one direction, the end tool 100 is also rotated in a direction intuitively the same as the one direction, so that the pitch motion or yaw motion is performed. Of course, “the same direction” as used herein may not be a perfectly matching direction on a three-dimensional coordinate, and may be understood to be equivalent to the extent that, for example, when the user's finger moves to the left, the end portion of the end tool 100 is moved to the left, and when the user's finger moves down, the end portion of the end tool 100 is moved down.

In the surgical instrument 10 according to the first embodiment of the present disclosure, the manipulation direction of the manipulation part 200 is ensured to be intuitively the same as the operation direction of the end tool 100, and to this end, portions of the manipulation part 200, which actually move for the actuation, yaw, and pitch motions, are formed to extend in the positive (+) X-axis direction from the rotation center of the corresponding joint of each motion, like the end tool 100.

Hereinafter, the power transmission part (not shown) of the surgical instrument 10 of FIG. 1 will be described in more detail.

The power transmission part of the surgical instrument 10 according to the first embodiment of the present disclosure may include one or more wires. In detail, the power transmission part may include a first jaw wire 301 that is connected to the manipulation part 200 and transmits the rotation of the manipulation part 200 to the first jaw 101, and a second jaw wire 302 that is connected to the manipulation part 200 and transmits the rotation of the manipulation part 200 to the second jaw 102.

Also, the power transmission part of the surgical instrument 10 according to an embodiment of the present disclosure may include a coupling member (not shown) which is coupled to each end portion of each wire in order to couple the wire to a pulley. Here, each of the coupling members may have various shapes as necessary, such as a ball shape, a tube shape, and the like.

When the coupling member (not shown) is coupled to the pulley, the wire may be fixedly coupled to the pulley. As such, the pully may be rotated as the wire is pulled and released.

Consequently, when the pulley of the manipulation part 200 is rotated by a motor or human force, the wire connected to the corresponding pulley is pulled and released, and accordingly, the pulley of the end tool 100 may be rotated.

Hereinafter, the end tool 100 of the surgical instrument 10 of FIG. 2 will be described in more detail.

Referring to FIGS. 1 and 2 and the like, the end tool 100 according to the first embodiment of the present disclosure includes a pair of jaws for performing a grip motion, that is, the first jaw 101 and the second jaw 102. Here, each of the first jaw 101 and the second jaw 102, or a component encompassing the first jaw 101 and the second jaw 102 may be referred to as the jaw 103.

In addition, the end tool 100 may include a pulley 111 associated with the rotation movement of the first jaw 101 and a pulley 121 associated with the rotation movement of the second jaw 102. Here, the pulley 111 and the pulley 121 may be referred to as end tool jaw pulleys, which will be described later.

Here, in the drawings, the pulleys facing each other are formed parallel to each other, but the concept of the present disclosure is not limited thereto, and each of the pulleys may be variously formed at a position and in a size suitable for the configuration of the end tool.

In addition, the end tool 100 according to the first embodiment of the present disclosure may include an end tool hub 180.

A rotation shaft 141 that will be described later is inserted through the end tool hub 180, and the end tool hub 180 may accommodate at least parts of the pulley 111 and the pulley 121 that are axially coupled to the rotation shaft 141. Here, the rotation shaft 141 may serve as an end tool jaw pulley rotation shaft.

The pulley 111 serves as an end tool first jaw pulley, and the pulley 121 serves as an end tool second jaw pulley. The pulley 111 may be also referred to as a first jaw pulley, the pulley 121 may be also referred to as a second jaw pulley, and these two components may be collectively referred to as end tool jaw pulleys.

The pulley 111 and pulley 121, which are the end tool jaw pulleys, are configured to face each other and are configured to rotate independently of each other around the rotation shaft 141 that is the end tool jaw pulley rotation shaft. Here, in the drawing, the pulley 111 and the pulley 121 are configured to rotate around one rotation shaft 141, but each of the pulleys may be configured to rotate around a separate shaft. Here, the first jaw 101 is fixedly coupled to the pulley 111 and rotates along with the pulley 111, and the second jaw 102 is fixedly coupled to the pulley 121 and rotates along with the pulley 121. The yaw motion and the actuation motion of the end tool 100 are performed according to the rotations of the pulley 111 and the pulley 121. That is, when the pulley 111 and the pulley 121 rotate in the same direction around the rotation shaft 141, the yaw motion is performed, and when the pulley 111 and the pulley 121 rotate in opposite directions around the rotation shaft 141, an actuation motion is performed.

Here, the first jaw 101 and the pulley 111 may be formed as separate members and coupled to each other, or the first jaw 101 and the pulley 111 may be formed as one-body. Likewise, the second jaw 102 and the pulley 121 may be formed as separate members and coupled to each other, or the second jaw 102 and the pulley 121 may be formed as one-body.

Meanwhile, the end tool 100 may further include an auxiliary pulley at sides of the pulley 111 and the pulley 121 that are the end tool jaw pulleys. That is, another rotation shaft may be provided on one side parallel to the rotation shaft 141 of the end tool hub, and the auxiliary pulley may be coupled to the rotation shaft and additionally provided at one side of the end tool jaw pulleys.

Hereinafter, various motions of the end tool 100 will be described in detail.

Referring to FIG. 2, the end tool 100 may perform an actuation motion. For example, the jaws of the end tool may perform a grip motion. In another example, the end tool 100 may perform a yaw motion. In another example, the end tool 100 may perform a pitch motion.

First, for describing a grip motion of the end tool 100, the first jaw 101 and the second jaw 102 may rotate as the angle therebetween changes along the rotation shaft 141. Here, the angle between the jaws may denote a relative angle formed by the first jaw 101 and the second jaw 102. That is, the angle between the jaws may be an angle formed by a first contact surface 1011 of the first jaw 101 and a second contact surface 1021 of the second jaw 102.

Also, a state in which the first jaw 101 and the second jaw 102 are in contact with each other may be expressed as ‘closed state’, and a state in which the first jaw 101 and the second jaw 102 form an angle exceeding 0° therebetween may be expressed as ‘open state’. From another perspective, the closed state may denote a state in which the first contact surface 1011 and the second contact surface 1021 are in contact with each other. Also, the open state may denote a state in which the first contact surface 1011 and the second contact surface 1021 are spaced apart from each other.

Also, in order for the first jaw 101 and the second jaw 102 to rotate, the end tool 100 may include the pulley 111 coupled to the first jaw 101 and the pulley 121 coupled to the second jaw 102, and the pulley 111 and the pulley 121 may rotate around the rotation shaft 141.

In addition, the end tool 100 may perform the grip motion within a predetermined range. Here, the predetermined range in which the grip motion is performed may be determined in various ways. For example, the predetermined range may be determined due to a fixedly coupled pulley-wire structure. In particular, the predetermined range may denote the rotation range of the first jaw 101 and the second jaw 102, in which an elastic deformation does not occur on the wire. That is, the predetermined range may denote a range in which the first jaw 101 and the second jaw 102 freely rotate. Alternatively, the predetermined range may be determined by a positional relationship between the first jaw 101/second jaw 102 and another component, for example, the end tool hub 180. That is, the predetermined range may be the rotation range of the first jaw 101 and the second jaw 102 until the first jaw 101 and the second jaw 102 come into contact with the end tool hub 180.

In particular, the first jaw 101 and the second jaw 102 according to the embodiment may rotate while the angle therebetween changes, within a grip range G1. Here, the grip range G1 may denote a range from a first grip boundary G2-1 to a second grip boundary G2-2. Here, the first grip boundary G2-1 and the second grip boundary G2-2 may define the boundaries of the rotation range of the first jaw 101 and the second jaw 102 during the grip motion. In an example, the first grip boundary G2-1 and the second grip boundary G2-2 may denote a point where the elastic deformation of the wire starts to occur due to the rotation of the pulley 111 and the pulley 121. In other words, it may be described that, in the range between the first grip boundary G2-1 and the second grip boundary G2-2, the first jaw 101 and the second jaw 102 freely rotate and the elastic deformation of the wire does not occur.

In addition, an angle formed by the first grip boundary G2-1 and the second grip boundary G2-2 may vary. In an example, the first grip boundary G2-1 and the second grip boundary G2-2 may form an angle of 180° with respect to each other. In particular, the first grip boundary G2-1 may form an angle of +90° with respect to the longitudinal axis of the straight part 401 (see FIG. 1), and the second grip boundary G2-2 may form an angle of −90° with respect to the longitudinal axis of the straight part 401 (see FIG. 1). In other words, the first jaw 101 and the second jaw 102 rotate until each forms ±90° with respect to the longitudinal axis of the straight portion (401, see FIG. 1) and perform the grip motion.

Also, the end tool 100 may perform a grip-press motion. Here, the grip-press motion may denote that the first jaw 101 and the second jaw 102 press each other while the end tool 100 is in the closed state. In detail, the grip-press motion may denote that the first jaw 101 and the second jaw 102 press each other with the force corresponding to an additional tension applied to the wire while the first jaw 101 and the second jaw 102 are in contact with each other. Here, the additional tension applied to the wire may be caused by the input applied to the manipulation part 200, and detailed description thereof is provided later.

Referring to FIG. 1 to FIG. 4, the manipulation part 200 of the surgical instrument 10 according to the first embodiment of the present disclosure includes a first handle 204 that the user may grip, the actuation manipulation part 203 controlling the actuation motion of the end tool 100, a yaw manipulation part 202 controlling the yaw motion of the end tool 100, and a pitch manipulation part 201 controlling the pitch motion of the end tool 100. Here, it may be understood that FIGS. 3 and 4 only show the components associated with the pitch/yaw/actuation motions of the surgical instrument 10.

The manipulation part 200 may include various pulleys associated with the rotation movement of the first jaw 101, including a pulley 210. Also, the manipulation part 200 may include various pulleys associated with the rotation movement of the second jaw 102, including a pulley 220. Also, the manipulation part 200 may include various pulleys associated with the pitch movement, and may include pulleys that are relay pulleys disposed in the middle of the bent part 402 of the connection part 400.

Here, in the drawings, the pulleys facing each other are formed parallel to each other, but the concept of the present disclosure is not limited thereto, and each of the pulleys may be variously formed at a position and in a size suitable for the configuration of the manipulation part.

Also, the manipulation part 200 according to the first embodiment of the present disclosure may include a rotation shaft 241 and a rotation shaft 242. Here, the rotation shaft 241 may serve as a first jaw actuation rotation shaft of the manipulation part, and the rotation shaft 242 may serve as a second jaw actuation rotation shaft of the manipulation part.

The pulley 210 may serve as a first jaw actuation pulley, the pulley 220 may serve as a second jaw actuation pulley, and these components may be collectively referred to as actuation pulleys.

Hereinafter, each component of the manipulation part 200 will be described in more detail.

The first handle 204 may be formed to be gripped by a user with the hand, and specifically, may be formed to be gripped by the user by wrapping the first handle 204 with his/her palm. In addition, the actuation manipulation part 203 and the yaw manipulation part 202 are formed on the first handle 204, and the pitch manipulation part 201 is formed at one side of the yaw manipulation part 202. In addition, the other end portion of the pitch manipulation part 201 is connected to the bent part 402 of the connection part 400.

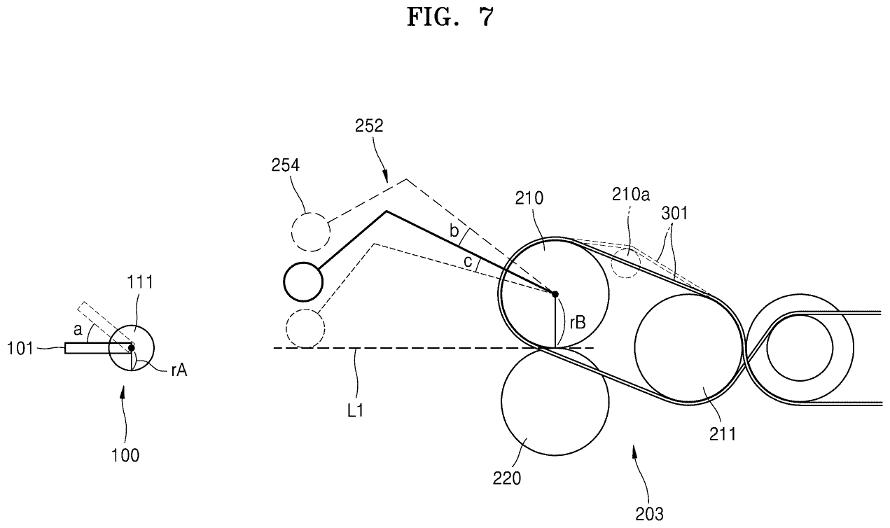

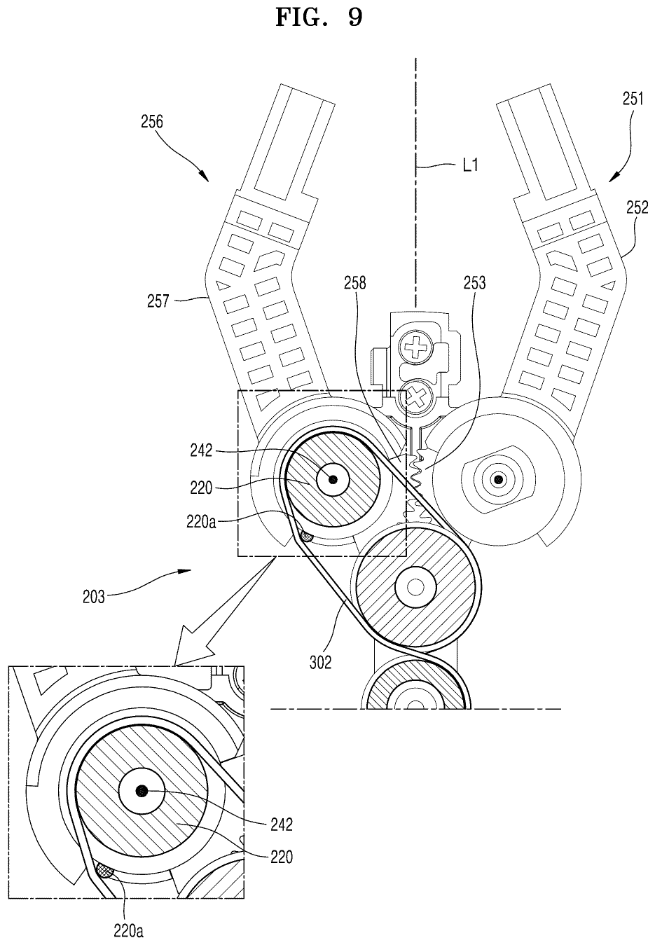

The actuation manipulation part 203 includes a first actuation manipulation part 251 and a second actuation manipulation part 256. The first actuation manipulation part 251 includes the rotation shaft 241, the pulley 210, a first handle member 252, a first tensioner 210a (see FIG. 7), and a first actuation gear 253. The second actuation manipulation part 256 includes the rotation shaft 242, the pulley 220, a second handle member 257, a second tensioner 220a (see FIG. 9), and a second actuation gear 258.

Here, a tensioner is a structure that rotates along with the actuation pulley and may come into contact with the wire according to the rotation of the actuation pulley and apply an additional tension to the wire. This will be described in detail later.

Also, the manipulation part 200 may further include a first ring member 254 and a second ring member 259 that are respectively connected to the first handle member 252 and the second handle member 257. Here, the first and second ring members 254 and 259 have the structures in which the fingers of the user may be inserted and seated and may have various shapes. For example, the first and second ring members 254 and 259 may be formed in hand ring shapes and may act as a second handle.

Here, the rotation shaft 241 and the rotation shaft 242 that are the actuation rotation shafts may be formed to form a certain angle with respect to an XY plane where the connection part 400 is formed. For example, the rotation shaft 241 and the rotation shaft 242 may be formed parallel to the Z-axis, and when the pitch manipulation part 201 or the yaw manipulation part 202 rotate in this state, a coordinate system of the actuation manipulation part 203 may relatively change. Of course, the concept of the present disclosure is not limited thereto, and according to an ergonomic design, the rotation shaft 241 and the rotation shaft 242 may be formed in various directions to be suitable for the hand structure of the user gripping the actuation manipulation part 203.

In addition, the pulley 210, the first handle member 252, and the first actuation gear 253 are fixedly coupled to one another and may be configured to rotate around the rotation shaft 241 along with one another. Here, the pulley 210 may include one pulley or may include two pulleys that are fixedly coupled to each other.

Likewise, the pulley 220, the second handle member 257, and the second actuation gear 258 may be fixedly coupled to one another and may be configured to rotate around the rotation shaft 242 along with one another. Here, the pulley 220 may include one pulley or may include two pulleys that are fixedly coupled to each other.

Here, the first actuation gear 253 and the second actuation gear 258 are configured to be engaged with each other, so that, when one of these rotates, the other also rotates in the opposite direction along with each other.

In addition, the first actuation rotation shaft 241 and the second actuation rotation shaft 242 may be formed on the first handle 204. Here, the first actuation rotation shaft 241 and the second actuation rotation shaft 242 are directly formed on the first handle 204, and thus, the first handle 204 and the actuation manipulation part 203 may be directly connected to each other.

Here, in the drawings, it is illustrated that the connection part 400 is formed to be curved with a predetermined curvature by having the bent part 402, but the concept of the present disclosure is not limited thereto, and the connection part 400 may be formed linearly or to be bent one or more times as necessary, and even in this case, it may be said that the pitch manipulation part 201 and the end tool 100 are formed on substantially the same axis or parallel axes. In addition, although FIG. 1 illustrates that each of the pitch manipulation part 201 and the end tool 100 is formed on an axis parallel to the X-axis, the concept of the present disclosure is not limited thereto, and the pitch manipulation part 201 and the end tool 100 may be formed on different axes.

### (Actuation Motion)

The actuation motion according to the embodiment is described as follows.

While the user inserts the index finger in the hand ring formed in the first handle member 252 and inserts the thumb into the hand ring formed in the second handle member 257, when the user rotates the handle members 252 and 257 by using one of the fingers or both fingers, the first actuation pulley 210 and the first actuation gear 253 fixedly coupled to the first handle member 252 rotate around the first actuation rotation shaft 241, and the second actuation pulley 220 and the second actuation gear 258 fixedly coupled to the second handle member 257 rotate around the second actuation rotation shaft 242. Here, the pulley 210 and the pulley 220 rotate in the opposite directions to each other, and thus, the first jaw wire 301 of which one end is fixedly coupled to the pulley 210 to be wound on the pulley 210 and the second jaw wire 302 of which one end is fixedly coupled to the pulley 220 to be wound on the pulley 220 also move in the opposite directions to each other. In addition, the above rotational force is transmitted to the end tool 100 via the power transmission part (not shown), and thus, two jaws 103 of the end tool 100 perform the actuation motion.

Here, as described above, the actuation motion denotes a motion of opening or closing the jaws 101 and 102 while the two jaws 101 and 102 rotate in opposite directions to each other. That is, when the handle members 252 and 257 of the actuation manipulation part 203 are rotated in a direction close to each other, the first jaw 101 rotates in a counter-clockwise direction and the second jaw 102 rotates in a clockwise direction so that the end tool 100 is closed. On the contrary, when the handle members 252 and 257 of the actuation manipulation part 203 are rotated in a direction away from each other, the first jaw 101 rotates in the clockwise direction and the second jaw 102 rotates in the counter-clockwise direction so that the end tool 100 is opened.

In the embodiment, the second handle is configured to have the first handle member 252 and the second handle member 257 for the above-mentioned actuation motion and may be manipulated by being gripped with two fingers. However, the configuration of the actuation manipulation part 203 for performing the actuation manipulation which opens and closes two jaws of the end tool 100 may include other modified examples, e.g., two actuation pulleys (pulley 210 and pulley 220) are configured to operate opposite to each other by using one actuation manipulation part, unlike the above description.

In summary, in the surgical instrument 10 according to the embodiment of the present disclosure, the pulleys are formed on respective joint points (an actuation joint, a yaw joint, and a pitch joint), the wires (the first jaw wire or the second jaw wire) are wound around the pulleys, and the rotational manipulations (an actuation rotation, a yaw rotation, and a pitch rotation) of the manipulation part cause the movement of respective wires, and consequently, the desired motion of the end tool 100 may be induced. Furthermore, the auxiliary pulley may be formed at one side of each of the pulleys, and the wire may not be wound several times around one pulley due to the auxiliary pulley.

Here, each pulley of the manipulation part 200 and each pulley of the end tool 100 form a corresponding relationship with each other. Here, the actuation pulley is described.

In detail, the pulley 111 which rotates around the jaw rotation shaft 141 and performs the yaw/actuation motions in the end tool 100 and the first actuation pulley 210 which rotates around the first actuation rotation shaft 241 and performs the actuation motion in the manipulation part 200 form a corresponding relationship with each other. In addition, the pulley 121 which rotates around the jaw rotation shaft 141 and performs the yaw/actuation motions in the end tool 100 and the second actuation pulley 220 which rotates around the second actuation rotation shaft 242 and performs the actuation motion in the manipulation part 200 form a corresponding relationship with each other.

Here, the pulley 111 and the pulley 121 of the end tool 100 are the pulleys performing the yaw motion described above, and at the same time, the pulleys performing the actuation motion.

As previously defined, the pulley 111 and the pulley 121 of the end tool 100 are grouped and referred to as the end tool jaw pulleys. In addition, for convenience of description, the first actuation pulley 210 and the second actuation pulley 220 of the manipulation part 200 are grouped and referred to as manipulation part actuation pulleys.

In summary, when the manipulation part actuation pulley rotates around the first actuation rotation shaft 241 and/or the second actuation rotation shaft 242, the end tool jaw pulley performs the actuation motion while rotating around the jaw rotation shaft 141.

Here, in the embodiment, (at least a part of) the end tool jaw pulley and (at least a part of) the manipulation part actuation pulley may be formed to have different diameters from each other. In addition, because (at least a part of) the end tool jaw pulley and (at least a part of) the manipulation part actuation pulley are formed to have different diameters from each other as described above, (at least a part of) the end tool jaw pulley and (at least a part of) the manipulation part actuation pulley may have different rotation angles from each other. Hereinafter, this will be described below in more detail.

When the manipulation part actuation pulley and the end tool jaw pulley are rotated along with each other, ‘lengths’ of the wires wound around the above pulleys change equally. Therefore, by adjusting the diameter of the pulley on which the wire is wound, when the wire moves the same length, the rotation angle of the pulley on which the wire is wound varies.

In particular, even when the manipulation part actuation pulley is rotated a little, the end tool jaw pulley corresponding to the manipulation part actuation pulley has to rotate by a greater angle, and thus, (at least a part of) the end tool jaw pulley may be formed to have a less diameter than that of (at least a part of) the manipulation part actuation pulley.

In an example with detailed numerical values, when the diameter of the end tool jaw pulley is φ5 and the diameter of the manipulation part actuation pulley is φ15, (diameter of the end tool jaw pulley: diameter of the manipulation part actuation pulley) may be 1:3. From another perspective, (the rotation angle of the manipulation part actuation pulley: the rotation angle of the end tool jaw pulley) may be 1:3.

As described above, by forming the diameters and the rotation angles of (at least a part of) the end tool jaw pulley and (at least a part of) the manipulation part actuation pulley to be different from each other, the user's convenience in manipulation is improved, and at the same time, precise and accurate manipulation level may be maintained.

Referring to FIG. 3, the manipulation part 200 may receive application of a user input and perform a grip motion. In detail, the first actuation manipulation part 251 and the second actuation manipulation part 256 may independently rotate around the first actuation rotation shaft 241 and the second actuation rotation shaft 242. Here, the first actuation manipulation part 251 and the second actuation manipulation part 256 may rotate as an angle therebetween changes. In detail, the first actuation manipulation part 251 includes a first handle member 252 operatively connected to the first actuation pulley 210, the second actuation manipulation part 256 includes the second handle member 257 operatively connected to the second actuation pulley 220, and the first actuation manipulation part 251 and the second actuation manipulation part 256 may rotate while an angle between the first handle member 252 and the second handle member 257 changes.

The user input may denote various inputs. For example, the user input may denote the force applied to the surgical instrument 10 by the hand of the user. Alternatively, the user input may denote an input signal applied to the manipulation part for driving a surgical robot.

Here, an angle formed by the first actuation manipulation part 251 and the second actuation manipulation part 256 according to the user input may be referred to as an input angle. That is, the input angle may be defined by a mutual angle relationship between the first handle member 252 and the second handle member 257.

Referring back to FIG. 3, the first handle member 252 and the second handle member 257 may be rotated respectively around the first actuation rotation shaft 241 and the second actuation rotation shaft 242 separately or along with each other on an XY plane (see FIG. 1). In FIG. 3, the first handle member 252 and the second handle member 257 are opened by an angle corresponding to a handle-press range G14, and an angle formed by one handle member with respect to the other handle member may be described as the input angle. Alternatively, as shown in FIG. 9, etc., an angle formed by the first handle member 252 based on a virtual line L1 that is parallel to a direction in which the connection part 400 extends (X-axis direction of FIG. 1) and is formed between the first actuation manipulation part 251 and the second actuation manipulation part 256, may be referred to as the input angle. Likewise, an angle formed by the second handle member 257 based on the virtual line L1 may be referred to as the input angle.

Also, when performing the grip motion, the first actuation manipulation part 251 and the second actuation manipulation part 256 may independently rotate within a first manipulation range G10-1 and a second manipulation range G10-2, respectively. Here, as the first actuation manipulation part 251 and the second actuation manipulation part 256 independently rotate within the first manipulation range G10-1 and the second manipulation range G10-2, respectively, the input angle may change. In other words, as the first actuation manipulation part 251 and the second actuation manipulation part 256 rotate within the first manipulation range G10-1 and the second manipulation range G10-2 due to the user input, the input angle may change.

Here, in the first manipulation range G10-1 and the second manipulation range G10-2, it may be described that the first actuation manipulation part 251 and the second actuation manipulation part 256 freely rotate and the elastic deformation of wire does not occur. As described above, as the input angle changes due to the user input, various motions of the end tool (see 100 of FIG. 1) may be implemented and will be described in detail later.

Referring back to FIG. 3, the manipulation part 200 may perform a handle-press motion. Here, the handle-press motion may denote that the first actuation manipulation part 251 and the second actuation manipulation part 256 further rotate within a handle-press range G14. In detail, the handle-press motion may denote that the first actuation manipulation part 251 and the second actuation manipulation part 256 further rotate by the rotation ranges of the first actuation pulley 210 and the second actuation pulley 220, the rotation ranges being increased by the elastic deformation caused on the wire outside the first and second manipulation ranges G10-1 and G10-2 that are determined in advance according to the fixedly coupled pulley-wire structure.

From another perspective, the handle-press motion may denote that the first actuation manipulation part 251 and the second actuation manipulation part 256 further rotate within the rotation range between first and second press boundaries G15-1 and G15-2. That is, it may denote that the first handle member 252 and the second handle member 257 further rotate in a direction closer to each other.

Here, the first and second press boundaries G15-1 and G15-2 may denote another boundaries of the grip motion and positions where the elastic deformation of the wire starts to occur. In addition, the handle-press range G14 may have a rotation range determined in advance so as to prevent plastic deformation of the wire. That is, the first actuation manipulation part 251 and the second actuation manipulation part 256 may be rotated while the input angle changes within the handle-press range G14 due to the user input.

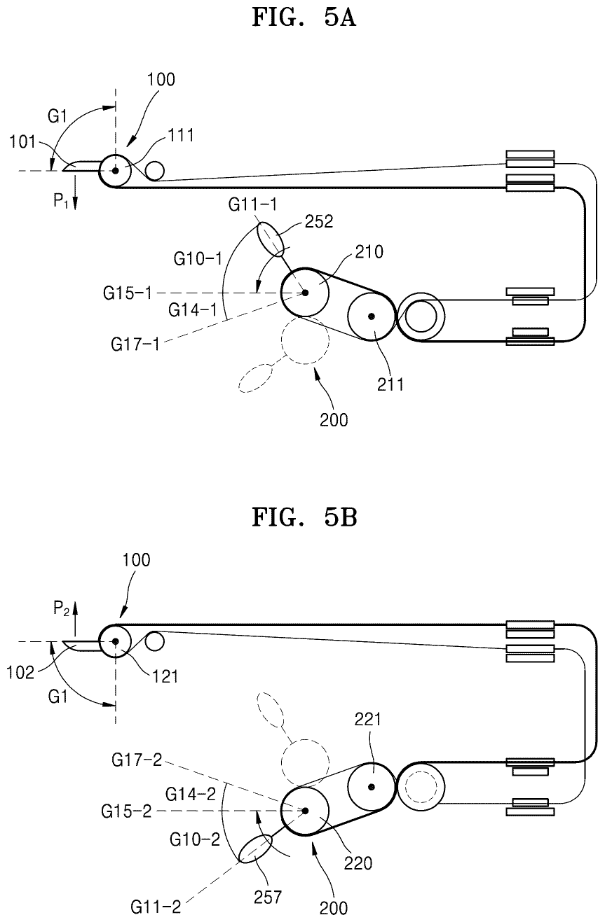

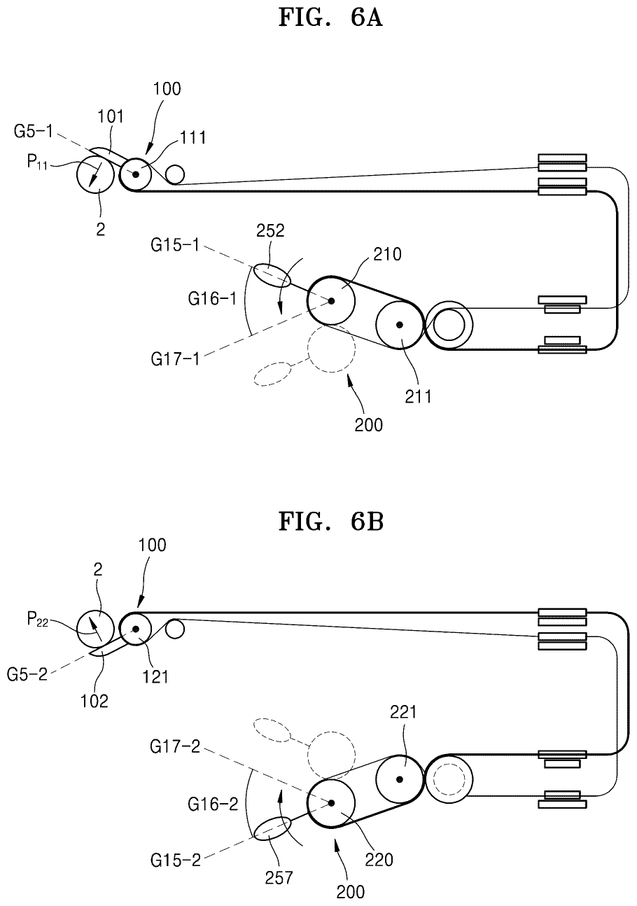

FIGS. 5A and 5B show a grip-press mechanism of the surgical instrument according to the first embodiment of the disclosure in view of the relationship between the manipulation part and the end tool, and FIGS. 6A and 6B show an object press mechanism of the surgical instrument according to the first embodiment of the present disclosure in view of the relationship between the manipulation part and the end tool.

Referring to FIGS. 5A and 5B, when the user input is applied to the manipulation part 200, the end tool 100 may perform a grip motion and a press motion. In detail, the first jaw 101 and the second jaw 102 may rotate in response to the rotation of the first actuation manipulation part 251 and the second actuation manipulation part 256 according to the user input. In more detail, when the first actuation manipulation part 251 and the second actuation manipulation part 256 respectively rotate within the first manipulation range G10-1 and the second manipulation range G10-2, the first jaw 101 and the second jaw 102 may rotate within the grip range G1 in response to the rotating angles of the first actuation manipulation part 251 and the second actuation manipulation part 256.

Here, when the first actuation manipulation part 251 and the second actuation manipulation part 256 are rotated up to the first and second press boundaries G15-1 and G15-2, the first jaw 101 and the second jaw 102 may be in the closed state. In the closed state, the first and second jaw pulleys 111 and 121 may no longer rotate.

In this state, when the first actuation manipulation part 251 and the second actuation manipulation part 256 further rotate respectively within the first and second handle-press ranges G14-1 and G14-2, the first jaw 101 and the second jaw 102 may press each other while being in contact with each other. In detail, the first jaw 101 and the second jaw 102 may press each other with forces P₁ and P₂ corresponding to the rotations of the first actuation manipulation part 251 and the second actuation manipulation part 256 within the first and second handle-press ranges G14-1 and G14-2.

In more detail, when the end tool 100 is in the closed state, the first and second jaw pulleys 111 and 121 that are operably connected respectively to the first jaw 101 and the second jaw 102 may not further rotate. In this state, when the first actuation manipulation part 251 and the second actuation manipulation part 256 are further rotated within the first and second handle-press ranges G14-1 and G14-2, while the first jaw pulley 111 and the second jaw pulley 121 are fixed, tension is applied to the wires connected to the first jaw pulley 111 and the second jaw pulley 121 due to the additional rotations of the first jaw pulley 111 and the second jaw pulley 121, thereby causing the elastic deformation of the wire. In addition, the first contact surface (see 1011 of FIG. 2) and the second contact surface (see 1021 of FIG. 2) press each other with the forces P₁ and P₂ corresponding to the tension applied to the wire as described above. Also, the first actuation manipulation part 251 and the second actuation manipulation part 256 may rotate to maximum press boundaries G17-1 and G17-2. Detailed descriptions thereof will be provided later.

Here, the rotation amounts of the first actuation manipulation part 251 and the second actuation manipulation part 256 within the first and second handle-press ranges G14-1 and G14-2 may correspond to the forces P₁ and P₂ applied to the first and second contact surfaces (see 1011 and 1021 of FIG. 2) of the end tool 100. Also, the rotation amounts of the first and second actuation manipulation parts 251 and 256 within the first and second handle-press ranges G14-1 and G14-2 may correspond to the elastic deformation amount of the wire.

When the first and second actuation manipulation parts 251 and 256 are rotated in the first and second grip ranges G10-1 and G10-2, the elastic deformation of the wire does not occur, but when the first and second actuation manipulation parts 251 and 256 are rotated within the first and second handle-press ranges G14-1 and G14-2, the elastic deformation of the wire occurs. Thus, the user input may be more strongly applied to the first actuation manipulation part 251 and the second actuation manipulation part 256 rotating within the first and second handle-press ranges G14-1 and G14-2.

That is, the grip force may increase during a maximum grip of the actuation manipulation part 203.

FIGS. 6A and 6B show an object press mechanism of the surgical instrument 10 according to the first embodiment of the present disclosure in view of a relationship between the manipulation part and the end tool.

Referring to FIGS. 6A and 6B, the end tool 100 may grip an object 2 in response to the motion of the manipulation part 200.

When the end tool 100 is in a state of gripping the object 2, the end tool 100 may press the object 2 when an additional user input is applied to the manipulation part 200. In detail, the first jaw 101 and the second jaw 102 may press the object 2 in response to the rotations of the first actuation manipulation part 251 and the second actuation manipulation part 256 due to the user input. In more detail, when the first actuation manipulation part 251 and the second actuation manipulation part 256 rotate in a first object press range G16-1 and a second object press range G16-2, respectively, the first jaw 101 and the second jaw 102 may press the object 2 with forces P₁₁ and P₂₂ corresponding to the rotations of the first actuation manipulation part 251 and the second actuation manipulation part 256.

The motion of the end tool 100 pressing the object 2 may be performed similarly to the motion of the first jaw 101 and the second jaw 102 pressing each other. That is, while the end tool 100 grips the object 2, the first jaw pulley 111 and the second jaw pulley 121 that are operably connected respectively to the first jaw 101 and the second jaw 102 may not rotate. In this state, when an additional force is applied due to the user input, the first and second actuation manipulation parts 251 and 256 may further rotate within the first and second object press ranges G16-1 and G16-2 while causing the elastic deformation of the wires that are operably connected to the first and second jaw pulleys 111 and 121. Consequently, the first contact surface (see 1011 of FIG. 2) and the second contact surface (see 1021 of FIG. 2) press the object 2 with the forces P₁₁ and P₂₂ corresponding to the tension applied to the wire due to the additional rotations of the first and second jaw pulleys 111 and 121. Also, the first actuation manipulation part 251 and the second actuation manipulation part 256 may rotate up to maximum press boundaries G17-1 and G17-2. Detailed descriptions thereof will be provided later.

Here, the rotation amounts of the first and second actuation manipulation parts 251 and 256 in the first and second object press ranges G16-1 and G16-2 and the forces P₁₁ and P₂₂ applied by the first and second contact surfaces (see 1011 and 1021 of FIG. 2) to the object 2 may be proportional to each other. Also, the rotation amounts of the first and second actuation manipulation parts 251 and 256 within the first and second object press ranges G16-1 and G16-2 may be proportional to the elastic deformation amounts of the wires.

Here, when the first and second actuation manipulation parts 251 and 256 are rotated in the first and second grip ranges (see G10-1 and G10-2 of FIGS. 5A and 5B), the elastic deformation of the wire does not occur, but when the first and second actuation manipulation parts 251 and 256 are rotated within the first and second object press ranges G16-1 and G16-2, the elastic deformation of the wire occurs. Thus, the user input may be more strongly applied to the first actuation manipulation part 251 and the second actuation manipulation part 256 rotating within the first and second object press ranges G16-1 and G16-2.

That is, the grip force may increase during a maximum grip of the actuation manipulation part 203.

Hereinafter, a tensioner included in the actuation manipulation part 203 of the surgical instrument 10 according to the first embodiment of the present disclosure will be described in detail below.

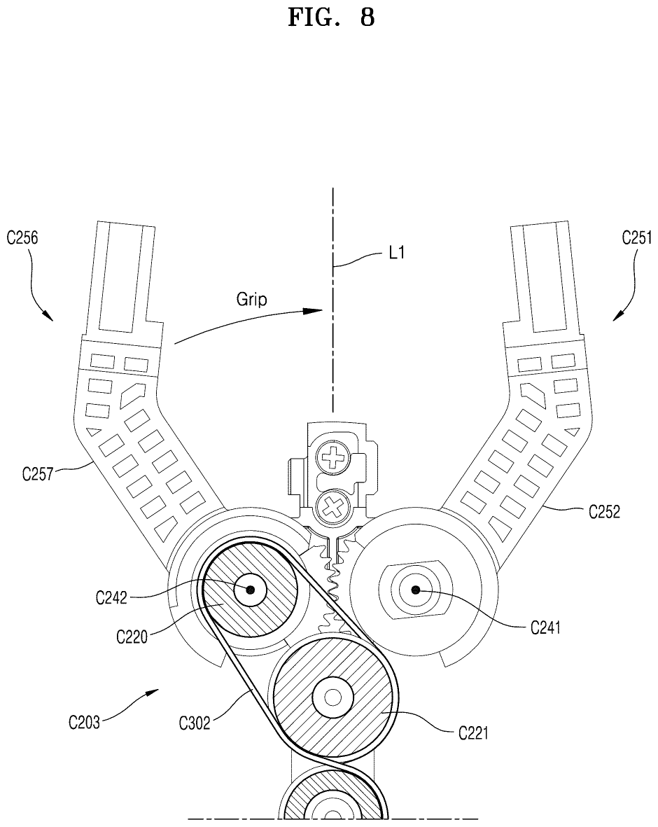

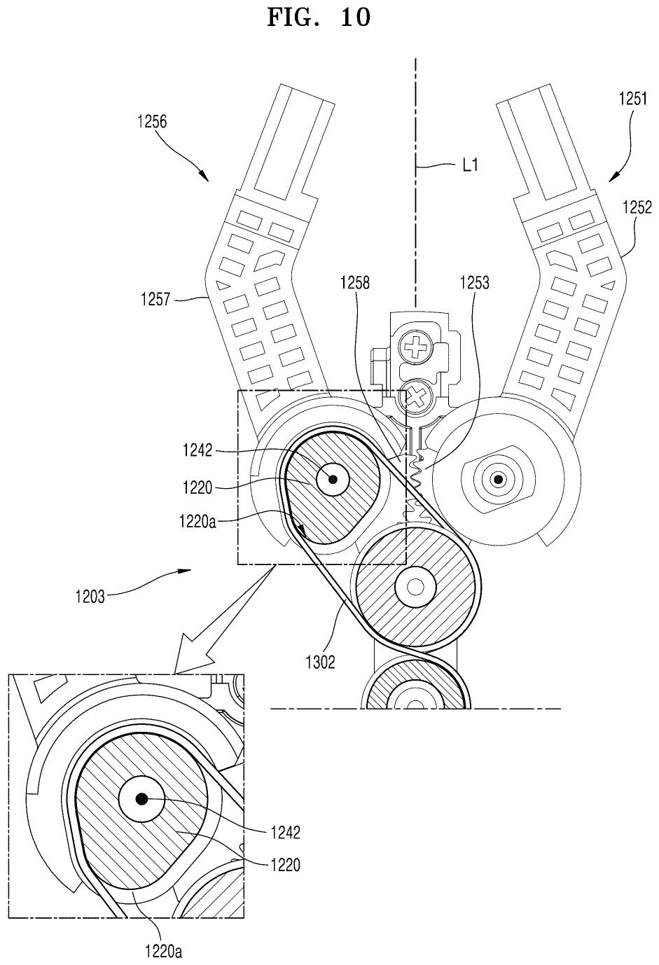

FIG. 7 is a diagram schematically showing the configuration for describing motions of the end tool and the manipulation part in the surgical instrument of FIG. 1, and FIG. 8 is a plan view showing a comparative technique for describing the actuation manipulation part of the surgical instrument according to the first embodiment of the present disclosure. FIG. 9 is a plan view of the actuation manipulation part in the surgical instrument according to the first embodiment of the present disclosure, and FIG. 10 is a plan view of an actuation manipulation part of a surgical instrument according to a modified example of the first embodiment of the present disclosure.

Referring to FIGS. 7 to 10, as described above, in the surgical instrument 10 according to the first embodiment of the present disclosure, the actuation manipulation part 203 includes a tensioner that rotates along with the rotation of the actuation pulley, and the tensioner may come into contact with the first jaw wire 301 or the second jaw wire 302 according to the rotation of the actuation pulley and may apply additional tension to the first jaw wire 301 or the second jaw wire 302.

In detail, in the first actuation manipulation part 251, the first actuation pulley 210 rotates around the first actuation rotation shaft 241, and the first handle member 252 may be also rotated. In addition, along with the rotations of the first actuation pulley 210 and the first handle member 252, a first tensioner 210a may rotate around the same rotation shaft, that is, the first actuation rotation shaft 241.

Likewise, in the second actuation manipulation part 256, the second actuation pulley 220 rotates around the second actuation rotation shaft 242, and the second handle member 257 may be also rotated. In addition, along with the rotations of the second actuation pulley 220 and the second handle member 257, a second tensioner 220a may rotate around the same rotation shaft, that is, the second actuation rotation shaft 242.

Operations and effects of the tensioner that is rotated along with the rotation of the actuation pulley as described above are described below with reference to FIG. 7. In other words, FIG. 7 is a plan view briefly showing the actuation manipulation part 203 and the end tool 100 of FIG. 1.

In addition, in order to easily describe the control of the actuation manipulation part 203 from the manipulation part 200 and the movement of the jaw 103 of the end tool 100, the relationship between the first actuation manipulation part 251 and the first jaw 101 of the end tool 100 is described as an example. Of course, the same description is applied to the relationship between the second actuation manipulation part 256 and the second jaw 102 of the end tool 100 within a corresponding range.

Referring to FIG. 7, the manipulation part 200 according to the first embodiment of the present disclosure may be provided with the first actuation pulley 210 and the second actuation pulley 220 of the actuation manipulation part 203, and a yaw pulley 211 at one side thereof. In addition, the first handle member 252 may be coupled to the first actuation pulley 210, and a first ring member 254 may be formed at the end portion of the first handle member 252 toward the end tool 100. Also, the first jaw wire 301 may be connected to the first actuation pulley 210 through the yaw pulley 211.

In addition, the end tool 100 may include the first jaw 101 that is rotated along with the pulley 111, and the end tool 100 may be located on the X-axis (See FIG. 1).

As described above, in the relationship between the actuation pulley and the end tool jaw pulley connected to each other via one or more wires, when the diameter of the actuation pulley is greater than that of the end tool jaw pulley, the rotation angle of the end tool jaw pulley may be greater than the rotation angle of the actuation pulley.

That is, when a radius of the first jaw pulley 111 is rA and a radius of the first actuation pulley 210 is rB, a rotation ratio of the pulley (rA) and the pulley (rB) is rB:rA. That is, when the radius rB of the first actuation pulley 210 is greater than the radius rA of the first jaw pulley 111, the first jaw 101 may greatly rotate even when the first actuation manipulation part 251 is less rotated.

As shown in FIG. 7, when the first handle member 252 coupled to the first actuation pulley 210 is rotated by angle b in a counter-clockwise direction, the first jaw 101 of the end tool 100 is rotated by angle a in the counter-clockwise direction. Here, the relationship between the rotation angles a and b may be expressed as a:b=rB:rA.

Here, the rotation of the first jaw 101 by the angle a due to the rotation of the first handle member 252 by the angle b may denote that the first jaw 101 is rotated within the grip range and is in the closed state. From another perspective, it may be described that the first handle member 252 is rotated within the first manipulation range G10-1 up to the first press boundary G15-1.

When the first actuation manipulation part 251 does not include the first tensioner 210a, the first jaw wire 301 passing the first actuation pulley 210 may be located on the same passage as that of the first jaw wire 301 expressed as a solid line in FIG. 7.

Here, when the first handle member 252 is further rotated by angle c in the counter-clockwise direction, the first jaw 101 of the end tool 100 may not move any further in the closed state. That is, because the additional rotation of the first jaw pulley 111 is caused in the fixed state in which the first jaw pulley 111 may not further rotate, tension is applied to the first jaw wire 301 connected to the first jaw pulley 111, thereby causing the elastic deformation of the wire. In addition, the first contact surface 1011 and the second contact surface 1021 press each other with the forces corresponding to the tension applied to the wire as described above.

In addition, the first actuation manipulation part 251 of the surgical instrument 10 according to the first embodiment of the present disclosure includes the first tensioner 210a, and thus, the first jaw wire 301 passing the first actuation pulley 210 may be located on the same passage as that of the first jaw wire 301 expressed in dashed lines of FIG. 7.

That is, when the first actuation manipulation part 251 has an input angle greater than a first angle determined in advance, the first tensioner 210a is not in contact with the first jaw wire 301, and when the first actuation manipulation part 251 has an input angle less than the first angle, the first tensioner 210a may be in contact with the first jaw wire 301.

From another perspective, it may be described that, when the first actuation manipulation part 251 has the input angle that is greater than or equal to the first angle determined in advance, the first jaw wire 301 is not elastically deformed, and when the first actuation manipulation part 251 has the input angle less than the first angle determined in advance, the first jaw wire 301 may be elastically deformed.

In other words, because a distance from the rotation shaft 241 to the first tensioner 210a is greater than the radius of the first actuation pulley 210, when the first actuation pulley 210 rotates, the first jaw wire 301 is pushed outward to be away from the first actuation pulley 210 so that the passage of the wire is changed, and the additional tension may be applied to the wire.

From another perspective, it may be described that, due to the first tensioner 210a, the passage through which the first jaw wire 301 passes increases and the wire is pulled as much as the increased length. That is, it may be described that the tension of the same degree as that of the rotation amount of the pulley according to the related art may be applied to the wire while reducing the rotation amount of the first actuation pulley 210.

In addition, when the rotation angle of the end tool first jaw pulley 111 according to the rotation of the first actuation pulley 210 in a state in which the first tensioner 210a comes into contact with the first jaw wire 301 is defined as a first rotation angle and the rotation angle of the end tool first jaw pulley 111 according to the rotation of the first actuation pulley 210 in a state in which the first tensioner 210a is spaced apart from the first jaw wire 301 is defined as a second rotation angle, the first rotation angle may be greater than the second rotation angle.

From another perspective, when the first tensioner 210a comes into contact with the first jaw wire 301 according to the rotation of the first actuation pulley 210, there is the same effect as that when the first jaw wire 301 is wound around the actuation pulley having greater diameter. Therefore, the rotation angle of the end tool first jaw pulley 111 according to the rotation of the first actuation pulley 210 in a state in which the first tensioner 210a is in contact with the first jaw wire 301 is greater than the rotation angle of the end tool first jaw pulley 111 according to the rotation of the first actuation pulley 210 in a state in which the first tensioner 210a is spaced apart from the first jaw wire 301. In other words, from a point in time when the first tensioner 210a comes into contact with the first jaw wire 301, the rotation angle of the first jaw pulley 111 according to the rotation of the first actuation pulley 210 may be further increased.

That is, it may be described that the purpose of the present disclosure is achieved by varying the movement of the wire depending on the motion angle range, not by moving the wire operating the end tool with a constant ratio according to the motion angle of the hand ring.

Referring back to FIG. 7, when the first actuation manipulation part 251 does not include the first tensioner 210a as in the above example, the first actuation manipulation part 251 has to be rotated by the angle c up to the maximum press boundary G17-1.

That is, when the first actuation manipulation part 251 is further rotated in the counter-clockwise direction according to the grip-press mechanism so that the first and second jaws 101 and 102 press each other, the first actuation manipulation part 251 has to be rotated by the angle c in order to rotate up to the range corresponding to the maximum press boundary G17-1.

On the other hand, when the first actuation manipulation part 251 includes the first tensioner 210a, the force corresponding to the rotation of the first actuation manipulation part 251 by the angle c may be applied even when the first actuation manipulation part 251 is only rotated by an angle less than the angle c.

In other words, the rotation amount that is necessary to apply the force corresponding to the rotation degree of the first actuation manipulation part 251 up to the maximum press boundary G17-1 is reduced, and thus, it denotes that the first actuation manipulation part 251 may be rotated only by a small angle.

In addition, the maximum press boundary G17-1 may denote a boundary of a range in which the first actuation manipulation part 251 is rotated within the press range up to the point in time when the plastic deformation starts to occur on the wire. Here, because the actuation manipulation part may not further rotate when the first actuation manipulation part 251 and the second actuation manipulation part 256 come into contact with each other, the plastic deformation of the wire may be prevented. Also, the positions where the first actuation manipulation part 251 and the second actuation manipulation part 256 are in contact with each other may be set as the maximum press boundaries G17-1 and G17-2 so as to prevent the plastic deformation of the wire.

As described above, the angle (e.g., angle c) of the range in which the first actuation manipulation part 251 may rotate up to the maximum press boundary G17-1 may be referred to as a margin angle.

Here, the surgical instrument 10 according to the first embodiment of the present disclosure may minimize the margin angle range and transfer the tension equally or additionally to the wire via the tensioner, aside from the reduction in the margin angle range. That is, the surgical instrument 10 according to the first embodiment of the present disclosure may amplify the grip force during the maximum grip of the actuation manipulation part 203. As such, usability of a surgical operator may be improved and the surgery may be smoothly performed.

As described above, the description about the first actuation manipulation part 251 may be equally applied to the second actuation manipulation part 256 to a corresponding extent. In addition, when the above is described by using the input angle formed between the first actuation manipulation part 251 and the second actuation manipulation part 256, that is, the input angle defined in view of the relationship between the first handle member 252 and the second handle member 257, in order to prevent the plastic deformation of at least one of the first jaw wire 301 and the second jaw wire 302 when the actuation manipulation part 203 has the input angle less than the first angle determined in advance, the input angle may be greater than or equal to a second angle determined in advance, and the second angle may be formed while the first handle member and the second handle member are in contact with each other.

Referring to FIGS. 8 to 10, an actuation manipulation part C203 of FIG. 8 does not include a tensioner in an actuation pulley, the actuation manipulation part 203 of FIG. 9 includes the second tensioner 220a, and an actuation manipulation part 1203 of FIG. 10 includes a second tensioner 1220a.

As shown in FIG. 9, the second tensioner 220a of the surgical instrument 10 according to the first embodiment of the present disclosure is configured to be rotated along with the second actuation pulley 220, and in an example, the second tensioner 220a may be formed on the second handle member 257. In detail, the second tensioner 220a protrudes from one surface of the second handle member 257, which faces the second actuation pulley 220, toward a virtual plane on which the second actuation pulley 220 is arranged, and may be formed in a lengthwise direction that is parallel to the second actuation rotation shaft 242. In detail, when an inner side surface of the second jaw wire 302, which comes into contact with the second actuation pulley 220, is defined as an inner surface of the second jaw wire 302, the second tensioner 220a may be formed adjacent to the second actuation pulley 220 so as to come into contact with the inner surface of the second jaw wire 302.

Likewise, although not shown in the drawing, the first tensioner 210a may be configured to rotate along with the first actuation pulley 210, and like the second tensioner 220a formed on the second handle member 257, the first tensioner 210a may be formed on the first handle member 252.

As described above, the handle member is fixedly coupled to the actuation pulley and is rotated along with the actuation pulley, and thus, the tensioner formed on the handle member may be rotated along with the actuation pulley. That is, according to the rotation of the actuation pulley, the tensioner comes into contact with the wire and changes the passage of the wire while pushing the wire outward so that the wire is spaced apart from the actuation pulley, and may apply additional tension to the wire.

In addition, the second tensioner 220a of a surgical instrument 10 according to a modified example of the first embodiment of the present disclosure may be fixedly coupled to the second actuation pulley 220. Also, the second tensioner 220a may be integrally formed with the second actuation pulley 220.

For example, as shown in FIG. 10, the second actuation pulley 1220 may be a non-circular pulley. That is, a part of the side surface of the pulley, on which the wire is wound, may protrude so as to have a radius greater than that of the other part in the side surface of the pulley. As described above, the protruding part of a non-circular pulley 1220 may act as a second tensioner 1220a. Likewise, although not shown in the drawing, the first actuation pulley 210 may be formed as a non-circular pulley, and a protruding part of the pulley may act as a first tensioner 1210a. That is, according to the rotation of the actuation pulley, the tensioner comes into contact with the wire and changes the passage of the wire while pushing the wire outward so that the wire is spaced apart from the actuation pulley, and may apply additional tension to the wire.

As described above, the surgical instrument according to the embodiment of the present disclosure may form a pully-wire mechanism that may apply additional force with respect to the rotation of the end tool jaw pulley according to the rotation of the actuation manipulation part, by adding the tensioner to the actuation manipulation part. Accordingly, the grip force during the maximum grip of the actuation manipulation part may be amplified, thereby improving the usability of the surgical operator and allowing the surgery to be smoothly performed.

While the present disclosure has been particularly shown and described with reference to preferred embodiments thereof, it will be understood by those skilled in the art that various changes in form and details may be made therein without departing from the spirit and scope of the present disclosure as defined by the appended claims. The preferred embodiments should be considered in descriptive sense only and not for purposes of limitation. Therefore, the scope of the present disclosure is defined not by the detailed description of the present disclosure but by the appended claims, and all differences within the scope will be construed as being included in the present disclosure.

According to the present disclosure, the surgical instrument may improve usability of the surgical operator and allow the surgery to be smoothly performed, by maintaining the motion integrity between the manipulation part and the end tool and amplifying the grip force during the maximum grip.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A surgical instrument comprising:
an end tool including a first jaw and a second jaw that are rotatable;
a manipulation part including an actuation manipulation part configured to control actuation motions of the first jaw and the second jaw, the manipulation part being configured to control motions of the end tool;
a power transmission part comprising:
a first jaw wire connected to the manipulation part to transfer a rotation of the manipulation part to the first jaw; and
a second jaw wire connected to the manipulation part to transfer the rotation of the manipulation part to the second jaw,
wherein the actuation manipulation part comprises:
an actuation pulley rotatable around an actuation rotation shaft; and
a handle member that is fixedly coupled to the actuation pulley, the handle member being rotatable along with the actuation pulley; and
a tensioner configured to rotate along with the rotation of the actuation pulley,
wherein the tensioner is configured to come into contact with the first jaw wire or the second jaw wire according to the rotation of the actuation pulley to apply additional tension to the first jaw wire or the second jaw wire.

2. The surgical instrument of claim 1,
wherein the tensioner is fixedly coupled to the actuation pulley.

3. The surgical instrument of claim 1,
wherein the tensioner and the actuation pulley are integrally formed, and the actuation pulley is a non-circular pulley.

4. The surgical instrument of claim 1,
wherein the tensioner is disposed on the handle member.

5. The surgical instrument of claim 1,
wherein the end tool further includes an end tool jaw pulley that is coupled to the first jaw or the second jaw, the end tool jaw pulley being rotatable around a jaw rotation shaft,
wherein the end tool jaw pulley and the actuation pulley are connected to each other via at least one jaw wire of the first jaw wire and the second jaw wire,
wherein the end tool jaw pulley has a diameter that is less than a diameter of the actuation pulley, and
wherein, when the at least one jaw wire moves, a rotation angle of the end tool jaw pulley is greater than a rotation angle of the actuation pulley.

6. The surgical instrument of claim 5,
wherein the rotation angle of the end tool jaw pulley according to the rotation of the actuation pulley in a state in which the tensioner comes into contact with the first jaw wire or the second jaw wire is defined as a first rotation angle,
wherein the rotation angle of the end tool jaw pulley according to the rotation of the actuation pulley in a state in which the tensioner is spaced apart from the first jaw wire or the second jaw wire is defined as a second rotation angle, and
wherein the first rotation angle is greater than the second rotation angle.

7. The surgical instrument of claim 1,
wherein the actuation manipulation part comprises:
a first actuation manipulation part including:
a first actuation pulley rotatable around a first actuation rotation shaft; and
a first handle member fixedly coupled to the first actuation pulley and rotatable along with the first actuation pulley; and
a second actuation manipulation part including:
a second actuation pulley rotatable around a second actuation rotation shaft; and
a second handle member fixedly coupled to the second actuation pulley and rotatable along with the second actuation pulley.

8. The surgical instrument of claim 7,
wherein the end tool is in a closed state when the first jaw and the second jaw are in contact with each other and in an open state when the first jaw and the second jaw are spaced apart from each other,
wherein the actuation manipulation part is configured to form an input angle defined as an angle relationship between the first handle member and the second handle member,
wherein, when the actuation manipulation part has the input angle greater than or equal to a first predetermined angle, the end tool is configured to be in the open state, and
wherein, when the actuation manipulation part has the input angle less than the first predetermined angle, a pressure between the first jaw and the second jaw is changed.

9. The surgical instrument of claim 8,
wherein, when the actuation manipulation part has the input angle greater than the first predetermined angle, the tensioner is configured to be spaced apart from the first jaw wire or the second jaw wire, and
wherein, when the actuation manipulation part has the input angle less than the first predetermined angle, the tensioner is configured to contact the first jaw wire or the second jaw wire.

10. The surgical instrument of claim 8,
wherein, when the actuation manipulation part has the input angle that is greater than or equal to the first predetermined angle, at least one of the first jaw wire and the second jaw wire is configured to remain in an elastically original state, and
wherein, when the actuation manipulation part has the input angle that is less than the first predetermined angle, at least one of the first jaw wire and the second jaw wire is elastically deformed.

11. The surgical instrument of claim 10,
wherein, when the actuation manipulation part has the input angle less than the first predetermined angle, the input angle is greater than or equal to a second predetermined angle to prevent plastic deformation of at least one of the first jaw wire and the second jaw wire.

12. The surgical instrument of claim 11,
wherein the first handle member and the second handle member are configured to contact each other to form the second predetermined angle.

13. The surgical instrument of claim 8,
wherein a force applied to the first jaw wire and the second jaw wire when the actuation manipulation part has the input angle less than the first predetermined angle is greater than a force applied to the first jaw wire and the second jaw wire when the actuation manipulation part has the input angle greater than the first predetermined angle.

14. The surgical instrument of claim 8,
wherein the end tool is further configured to have a grip state in which a first contact surface of the first jaw and a second contact surface of the second jaw are in contact with an external object,
wherein, when the actuation manipulation part has the input angle that is greater than or equal to a third angle, the end tool is in the released grip state, and an angle between the first contact surface and the second contact surface is changed due to a rotation of at least one of the first handle member and the second handle member, and
wherein, when the actuation manipulation part has the input angle less than the third angle, the end tool is configured to maintain the grip state and a pressure between the first contact surface and the second contact surface and the external object is changed due to a user input causing a rotation of at least one of the first handle member and the second handle member.
